(19) United States
(12) Patent Application Publication
(10) Pub. No.: US 2026/0151062 A1
(43) Pub. Date: Jun. 4, 2026

(54) METHOD AND SYSTEM FOR SELF-ADMINISTRATED SURVEILLANCE OF USE OF ADDICTIVE STIMULUS

(71) Applicant: KONTIGO CARE AB, Uppsala (SE)

(72) Inventors: Markku HÄMÄLÄINEN, UPPSALA (SE); Andreas ZETTERSTRÖM, MÅNKARBO (SE); Karl ANDERSSON, Vänge (SE); Mats WALLDEN, Uppsala (SE)

(21) Appl. No.: 19/122,398

(22) PCT Filed: Oct. 27, 2023
(86) PCT No.: PCT/SE2023/051071

§ 371 Date: Apr. 18, 2025

(30) Foreign Application Priority Data
Oct. 28, 2022 (SE) 2251252-9

(51) Int. Cl. A61B 5/16; A61B 3/113; A61B 5/00

(52) U.S. Cl. CPC A61B 5/163; A61B 3/113; A61B 5/0022; A61B 5/4845

(57) ABSTRACT

A method for self-administrated surveillance of use of addictive-stimulus for an individual comprises providing (S10) of a body-measurement schedule for the individual. The individual is requested (S20) to perform measurements of bodily conditions, comprising eye conditions analysable from camera recordings, within each of multiple measurement time-slots. Data of the measurements a respective measurement time are collected (S30) in a handheld user interaction device. The collected data is transmitting (S40) from the user interaction device and is stored (S50) in a central server. Likelihood information of that the individual was exposed to an addictive stimulus is estimated (S60), based on the collected data. The estimating comprises comparison with individualized baseline data associated with the individual. An addictive-stimulus use-discouraging action is initiated (S70) as a response to likelihood information being larger than a predetermined threshold. A system for self-administrated surveillance of use of addictive-stimulus for an individual is also disclosed.

### TECHNICAL FIELD

The present technology refers in general to drug-use surveillance, and in particular to self-administrated surveillance of use of addictive stimulus.

### BACKGROUND

In conventional drug testing, an individual is requested to provide a test at a specific time point. The nature of the currently available drug tests is such that they can only detect drug use that has occurred recent 1-3 days. Hence, an individual who is aware of an upcoming drug test can intentionally avoid the use of drugs just a few days prior to the test occasion and hence provide a misleading clean test. For practical reasons and cost, conventional drug testing, alcohol excluded, is often limited to e.g. biweekly or monthly tests, giving the individual ample opportunity to use drugs between scheduled tests without being caught.

Care systems for monitoring patients with substance use disorder is known, e.g. from the published European patent application EP 3721435 A1. This system is commercially available and implemented in the context of alcohol. In this implementation, the system uses a definite measurement system, a breathalyzer, which directly detects alcohol. This is discussed by Zetterström and co-authors in “The Clinical Course of Alcohol Use Disorder Depicted by Digital Biomarkers”, in Front Digit Health. 2021 Dec. 7; 3:732049. doi: 10.3389/fdgth.2021.732049. eCollection 2021. However, corresponding definite measurement systems are not available for other types of drugs.

The use of pupillometry for assessing if it is likely that an individual has consumed drugs is well known, as for example discussed by Pinheiro and da Costa in “Pupillary light reflex as a diagnostic aid from computational viewpoint: A systematic literature review”, in Journal of Biomedical Informatics 117 (2021) 103757. Pupillometry is, however, an indirect measurement and it does not unambiguously confirm that a drug in fact has been consumed. The published international patent application WO 2021/037788 A1 also discuss pupillometry in the field of drugs and substance abuse.

The disclosure “Illicit drugs: Effects on eye” as published by Deepika Dhingra, Savleen Kaur, and Jagat Ram in Indian J Med Res. 2019 September; 150(3): 228-238. (doi: 10.4103/ijmr.IJMR_1210_17) describes in general terms the effect of drugs belonging to different families in eye function.

In the book “Drug Abuse Handbook” (ISBN 0-8493-2637-0, I. Karch, Steven B., 1997) chapter 4.3, a detailed description of how different drugs affect eye reactions as seen in pupillometry is provided. Chapter 4.3.6 concludes that “The application of pupillometry for the detection of drugs of abuse is theoretically possible but the practical utility is limited.”, “Because of the large between-subject variation in pupillary measures, one must know the baseline values for the tested subject.”, “The profound influence of ambient light on pupillary measures dictates that the conditions under which measures are made be carefully controlled.”, and “Finally, the magnitude of the effects of the drugs studied are small and transient and often do not exceed the within-subject variability.”.

There is still a need for processes and systems allowing for a more reliable self-administrative monitoring of individuals having a known problem of use of addictive stimulus.

### SUMMARY

A general object of the present technology is to provide concepts increasing the accuracy of identifying relapse into addictive-stimulus use of an individual.

The above object is achieved by methods and devices according to the independent claims. Preferred embodiments are defined in dependent claims.

In general words, in a first aspect, a method for self-administrated surveillance of use of addictive-stimulus for an individual comprises providing of a body-measurement schedule for the individual. The body-measurement schedule comprises multiple measurement time-slots. The individual is requested to perform measurements of bodily conditions within each of the multiple measurement time-slots. The bodily conditions comprise eye conditions analysable from camera recordings. Data of each of the measurements of bodily conditions and a respective time when the measurements of bodily conditions were performed are collected in a handheld user interaction device. The collected data is transmitting from the user interaction device to a central server. The collected data is stored in the central server. Likelihood information of that the individual was exposed to an addictive stimulus is estimated, based on at least the collected data. The estimating comprises comparison of the measurements of bodily conditions with individualized data associated with said individual, said individualized base line data comprising data of said bodily conditions when being controlled unexposed to the addictive stimulus. An addictive-stimulus use-discouraging action is initiated as a response to data of the likelihood information being larger than a predetermined threshold.

In a second aspect, a system for self-administrated surveillance of use of addictive-stimulus for an individual, comprising a central server and a handheld user interaction device, communicationally connected to each other. The central server is configured for providing, to the user interaction device, a body-measurement schedule for the individual. The body-measurement schedule comprises multiple measurement time-slots. The central server is configured for, by means of the user interaction device, requesting the individual to perform measurements of bodily conditions within each of the multiple measurement time-slots. The bodily conditions comprise eye conditions. The user interaction device has measurement means, comprising a camera, for collecting data analysable for obtaining each of the measurements of bodily conditions, and a timer for determining a respective time when the measurements of bodily conditions were performed. The user interaction device is configured for transmitting the collected data from the user interaction device to the central server. The central server is configured for receiving the collected data and storing the collected data in a memory. The central server comprises a processor configured for estimating likelihood information of that the individual was exposed to an addictive stimulus, based on at least the collected data. The estimating comprises comparison of the measurements of bodily conditions with individualized baseline data associated with said individual, said individualized base line data comprising data of said bodily conditions when being controlled unexposed to the addictive stimulus. The central server is configured for initiating an addictive-stimulus use-discouraging action as a response to data of the likelihood information being larger than a predetermined threshold.

One advantage with the proposed technology is that any possibility for an individual under self-administrated drug-use monitoring to hide drug use through planned use may be eliminated. If use of drugs anyway is suspected, suitable actions may be initiated.

Other advantages will be appreciated when reading the detailed description.

### BRIEF DESCRIPTION OF THE DRAWINGS

The invention, together with further objects and advantages thereof, may best be understood by making reference to the following description taken together with the accompanying drawings, in which:

FIG. 1 illustrates diagrams of measurements of pupil size of different individuals;

FIG. 2 illustrates a diagram of data for healthy volunteers measuring the ability to converge eyes;

FIG. 3 is a flow diagram of steps of an embodiment of a method for self-administrated surveillance of use of addictive-stimulus for an individual;

FIG. 4 illustrates schematically an embodiment of a system for surveillance of use of addictive-stimulus for an individual;

FIG. 5 illustrates a table illustrating relations between stimulus categories and bodily conditions;

FIG. 6 is a flow diagram of steps of an individualization procedure;

FIGS. 7 and 8 illustrates measurement data collected when individuals are requested to cross-eyes;

FIG. 9 illustrates measurement data collected when individuals are requested to cross-eyes, after an individualization procedure has been applied to data;

FIG. 10 illustrates a typical result from a pupillary light reflex measurement; and

FIG. 11 illustrates results from a pupillary light reflex measurement made by different individuals.

### DETAILED DESCRIPTION

Throughout the drawings, the same reference numbers are used for similar or corresponding elements.

Individuals having a known problem of addiction to drugs may voluntarily or as a part of an agreed detoxification treatment be introduced into a drug-use monitoring program. It is advantageous if such program can be self-administrated, since it requires less resources from the health care providers. However, self-administration always introduces a certain risk for untrustful behaviour. Drug addiction is strong and individuals that actually wants to become free of drug may anyway be tempted to continue to use them. If such an individual is a subject of a self-administrated drug-use monitoring, there might be a risk that the individual intentionally or pseudo-unconsciously tries to deceive the monitoring.

The term “drug” refers to a single compound or a combination of multiple compounds capable of intoxicating an individual to a level where the general status of said individual is affected. One non-limiting example of an intoxicating chemical compound is ethanol, more commonly known as alcohol, which is readily available to individuals in wine, beer, spirits and other beverages. Ethanol is intoxicating individuals to a level where many countries have a limit for the allowed amount of ethanol in the blood to drive a car legally. Other intoxicating chemical compounds include, but are not limited to: cannabinoids as for example available in cannabis, caffeine, MDMA (3,4-methylenedioxy-methamphetamine), cocaine, amphetamine, methamphetamine, psilocybin (for example found in “magic mushrooms”), LSD, opiates and opioids, tranquilizers like barbiturates, benzodiazepines and the similar, ketamine, amyl nitrite, mephedrone, mescaline, DMT for example as primary ingredient in ayahuasca, cathine and cathinone (khat), methylphenidate, fentanyl, GHB, ecstasy, narcolepsy medications, sleeping pills, anxiolytics, sedatives, cough suppressants, benzydamine, ephedrine, pseudoephedrine, dimethyltryptamine (DMT), 5-MeO-DMT, theobromine, kavalactones, myristicin, atropine, scopolamine, mitragynine, mitraphylline, 7-hydroxymitragynine, raubasine, valerian, lysergic acid amide (LSA, ergine), ibogaine, arecoline, rauwolscine, yohimbine, corynantheidine, psilocybin, psilocin, bufotenin, ibotenic acid, muscimol, antihistamines including but not limited to diphenhydramine, chlorpheniramine, orphenadrine and hydroxyzine, scopolamine, paracetamol (para-acetylaminophenol), non-steroidal anti-inflammatory drugs (NSAIDs) such as salicylates, hydrocodone, codeine, oxycodone, hydromorphone, carisoprodol, chloral hydrate, diethyl ether, ethchlorvynol, gabapentin, gamma-butyrolactone (GBL, a prodrug to GHB), gamma-hydroxybutyrate (GHB), glutethimide, ketamine, meprobamate, methaqualone, phenibut, pregabalin, propofol, nepetalactone, dimenhydrinate, hyoscyamine, dextromethorphan, dextromethorphan, chlorpheniramine, methoxetamine, phencyclidine, and nitrous oxide, to mention a few examples.

Monitoring of addiction to alcohol is relatively simple, since there are simple, non-expensive self-administrated direct tests available. However, when turning into other drugs, such as e.g. central depressive substances, cannabinoids, central stimulants or opioids, the direct and conclusive tests are typically expensive and may even be non-recommended for self-administration. Frequent direct drug tests, prohibiting the addict to use the drug at any time, are thus not feasible in most cases. However, as mentioned above, less frequent direct tests allow for planning of an individual to deceive the tests by scheduling the intake of drugs to periods when no tests are performed.

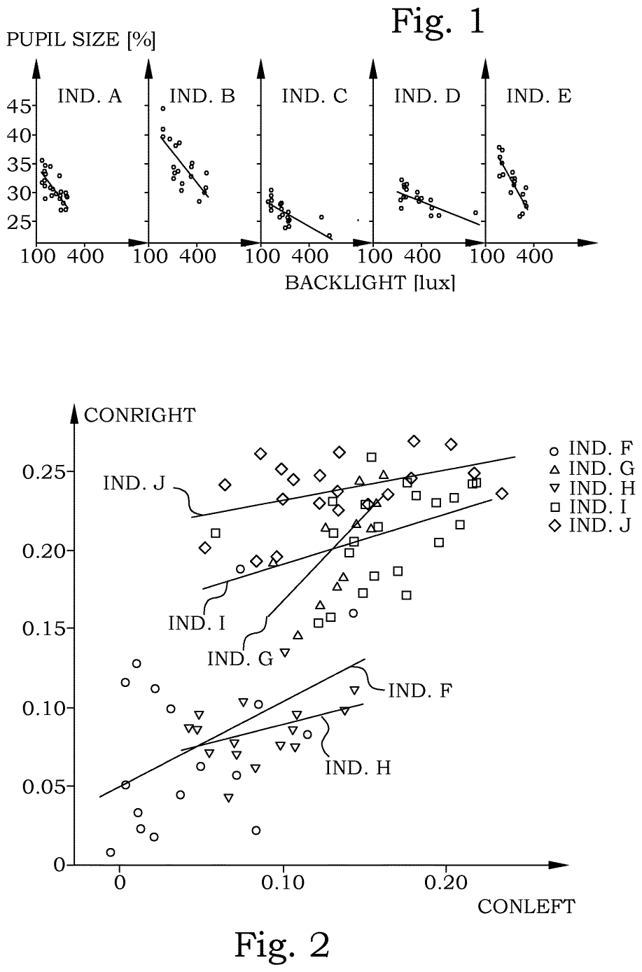

Indirect tests, such as e.g. pupillometry, are known to detect conditions that may be the result of drug intake. However, such measurements are up to now not considered as fully conclusive regarding the connection to drug intake. One common obstacle is the large variation between different individuals. FIG. 1 illustrates diagrams of measurements of pupil size of 5 different individuals Pupil size, expressed as pupil diameter in relation of iris diameter, was measured in different ambient light conditions. The diagrams show that the individuals have significantly differently sized pupils and that the pupil size changes in individual manners with the ambient light. In view of this, it is apparent that a single measurement, or even a series of measurements at a single occasion of an individual cannot conclusively distinguish between normal conditions and that extraordinary conditions are present.

Similar findings are relevant also for other types of eye measurements. Furthermore, in FIG. 2, a diagram is shown illustrating data for 5 healthy volunteers measuring the ability to converge eyes. Convergence of left eye, denoted CONLEFT, and right eye, denoted CONRIGHT, are compared. These positions are defined as position of the pupil relative to the eye centre when trying to cross the eyes. Volunteers F and H have significantly different abilities to cross eyes than the other volunteers. Also here, a single test or a series of tests at a single occasion will not be conclusive in any drug-related characterizations.

Moreover, even if it can be established that the eye conditions of an individual may differ from what is expected for this same individual, there is no direct one-to-one relationship with drug abuse. Many eye conditions that may react on drug use, may also react for other conditions, of the individual as well as of the surroundings. Physical or psychological fatigue, temperature, wind, emotional conditions etc. may give conditions similar to some of the ones caused by drug use. In order to finally establish that drugs in fact have been used, direct tests have to be performed.

As mentioned above, most drug tests may detect use of drugs only during a limited period of time after the consumption of the drug. This may also differ from one class of drugs to another. It is thus of interest that any drug tests should be performed as soon as possible after the drug is used and within a detection time window for the drug in question. Prescheduled drug tests with intervals longer than the detection time window are therefore of minor importance.

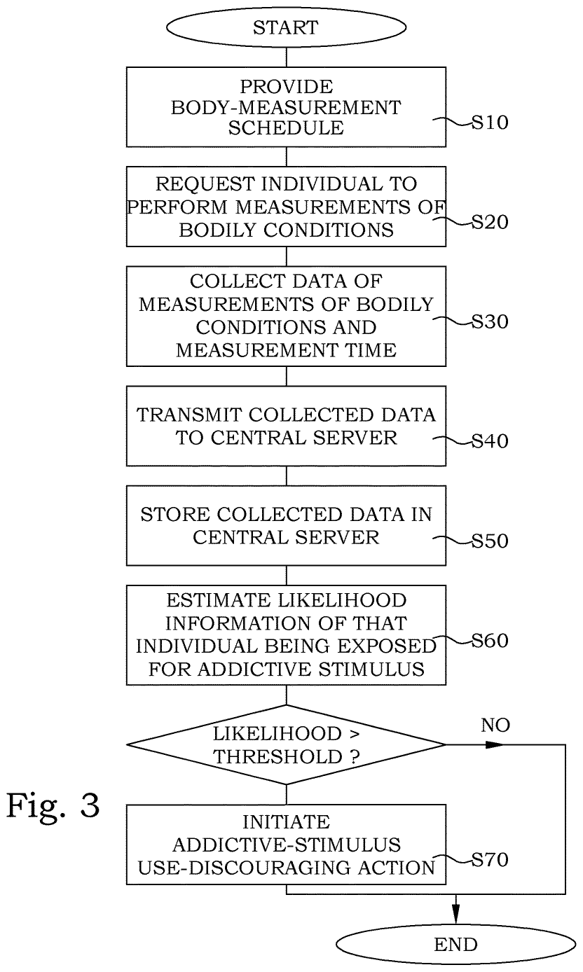

According to the present ideas, a two-stage procedure is instead proposed. FIG. 3 illustrates a flow diagram of steps of an embodiment of a method for self-administrated surveillance of use of addictive-stimulus for an individual. In step S10, a body-measurement schedule for the individual is provided. The body-measurement schedule comprises multiple measurement time-slots. This schedule is typically available for the individual, at least to a part. At least a next measurement time slot is preferably presented for the individual in connection with a previous measurement time slot, reducing the need for additional means for announcing a next measurement time slot by other means. However, the schedule may in particular embodiments be revised, e.g. depending on how and when the individual replies on requested measurement demands. In step S20, the individual is requested to perform measurements of bodily conditions within each of the multiple measurement time-slots. A typical way to implement this could be to send a message to be presented at the user interaction device. Other ways of communicating the request to the individual can of course also be utilized.

The bodily conditions according to the present ideas comprise eye conditions analysable from camera recordings. This typically comprise different visual conditions of the eye. Images or videos of the eyes of the individual may as indicated above contain image data that at least after an analysis may be connected to different types of eye conditions. Non-restricting examples may be pupil size, pupillary light reflex, behaviour of crossing eyes, nystagmus, saccadic eye movements and colour of eye whites. All these conditions are possible to deduce from images or videos of the eyes of the individuals, which is easily obtainable in a self-administrative manner. By using time stamping of the images and videos, the time for the recordings can be correctly established. Furthermore, these eye images may also be used for controlling the identity of the person on which the measurements are performed.

Preferably, the measurements of bodily conditions further comprise a recording of ambient light. As indicated above, the eye conditions may be dependent on the background illumination, and measurements may have to be further characterised by noticing at which ambient light conditions they are recorded.

Consequently, in step S30, data of each of the measurements of bodily conditions and a respective time when the measurements of bodily conditions were performed were collected in a user interaction device. In step S40, the collected data is transmitted from the user interaction device to a central server. In step S50, the collected data is stored in the central server. The typical timing for this is to transmit and store the data as soon as possible after each measurement. In other words, steps S30, S40 and S50 are performed in a sequence for each measurement time-slot. This ensures that the latest information regarding the individual will be present in the central server only a short while after the measurements are made.

The reaction magnitude for one individual may differ largely from the reaction for another individual. Hence, in order to detect a change in pupil reaction, it may be necessary to relate a measurement to the expected reaction to the identity of the individual. The estimating thereby comprises a comparison of the measurements of bodily conditions with individualized baseline data associated with the individual. This individualized baseline data comprises data of the bodily conditions associated with situations when a subject is controlled unexposed to the addictive stimulus.

The reaction magnitude for one individual may differ depending on the time at which the measurement is conducted. Typically, measurements conducted in the morning are more consistent, and measurements conducted in the afternoon are more variable and hence more difficult to individualize.

One way to obtain such individualized baseline data is to perform measurements on each individual. Thus in a preferred embodiment, the estimating comprises a comparison of the measurements of bodily conditions with individualized baseline measurements of the bodily conditions of the individual, recorded when the individual was controlled to be unexposed to the addictive stimulus. In other words, this in turn means that when an individual is starting to use the device, there could be a need for an introductory “calibration type” measurement procedure where the baseline characteristics of the individual is quantified. The need for an introductory “calibration type” measurement procedure also depends on which category of stimulus is being evaluated.

Alternatively, or as a complement, baseline data for different groups of individuals can be recorded in advance. This may be used e.g., if an individual is not guaranteed fully detoxicated at the first measurement occasion. Averages or typical measurements for the different groups can thereby be obtained, where the groups may be characterized e.g., according to age, gender, etc. When an individual is to start the surveillance, stored such typical data of a group associated with the age, gender etc. of the individual in question can be used as an approximation of an expected unexposed situation.

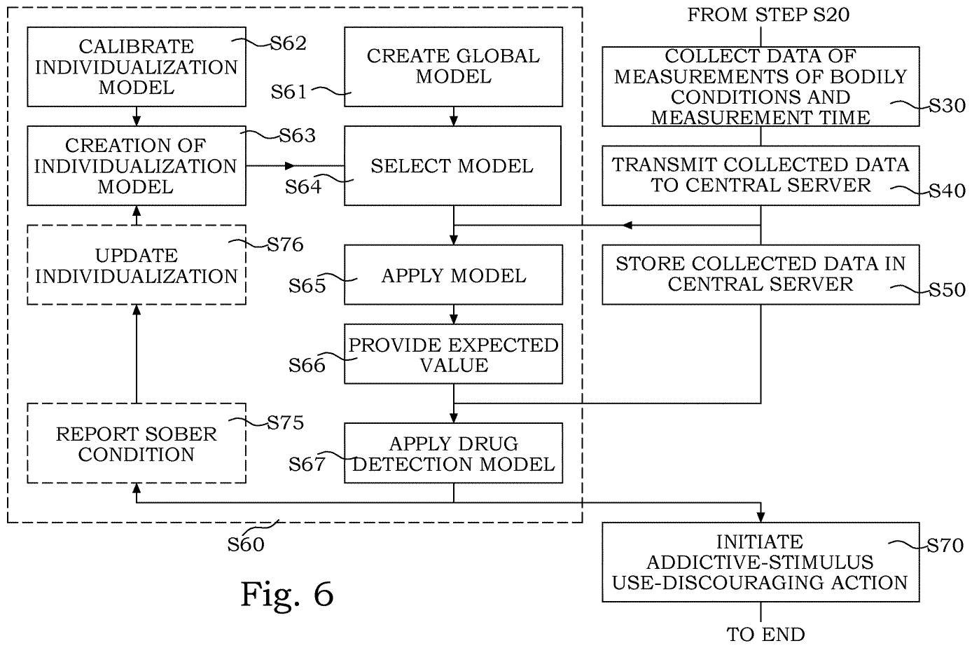

Referring now to FIG. 6 in which one non-limiting example of how individualization can be conducted is shown. The concept of individualization can be linked to a particular feature in measured data, for example the pupil size at the beginning of a measurement. Since the purpose of the individualization is to depict the typical value of the particular feature for an individual, and it is known that individuals are different, the individualization model must be calibrated in a first instance, as illustrated as S62. Calibration can for example be conducted in several known ambient light conditions or at several different times of the day, all at sober state. It is possible to conduct the individualization in a doctor's office, which increases the likelihood of the individual being sober during calibration and which allows direct addictive-stimulus testing using saliva or urine tests to confirm sobriety. The result of a calibration is the creation of an individualization model S63 that represents the expected value of the particular feature, possibly as a function of age, or ambient light level, or time of day, or any other similar boundary condition. Preferably, prior to use, the individualization model should be verified as functional by using quality control criteria, capable of distinguishing a good calibration from a bad.

With an approved individualization model, the calibration period ends. Now, upon making a measurement of the particular feature, i.e. collecting data of measurements of bodily conditions and measurement time S30, in a case where an individualization model exists, a model selector mechanism S64 can provide the individualization model to be applied S65, providing an expected value S66. An application of a drug detection model S67 has access to both the measured value, as transmitted S40 to the central server, and the expected value S66 of the particular feature. The application of the drug detection model S67 applies an algorithm to provided data S40, S66 for the purpose of determining if the measurement represents a sober condition or contains indications of drug use. A result indicating drug use initiates S70 an addictive-stimulus use-discouraging action, e.g. is reported to the individual him/herself or to health care providers or to relatives, as will be discussed further below. A result of a sober condition may also be reported S75 to the user. Optionally, upon registering a sober condition, the collected data can be fed back S76 to the individualization model to allow adjustment. With the feedback-loop active, the individualization model will become increasingly better, subject to the constraint that measurements deemed sober are indeed sober.

In cases where an individualization model is not available global model can be created S61. The model selector mechanism S64 can then provide the global model to the process of estimating an expected value for the particular feature under study.

A method for generating an individualization model associated with an individual can be provided as a part of a method for self-administrated surveillance of use of addictive-stimulus for an individual. However, such a method for generating an individualization model associated with an individual can also be provided as a separate method. In one embodiment, the individualization model comprising data of eye conditions analysable from camera recordings when being unexposed to any addictive stimulus. A calibration period is initiated by providing an eye-measurement schedule for the individual. The eye-measurement schedule comprises multiple measurement time-slots. The individual is requested to perform measurements of eye-conditions within each of the multiple measurement time-slots. Data is collected of each of the measurements of eye-conditions and, a respective time when the measurements of eye-conditions were performed in a handheld user interaction device. The individualization model 20 is generated based on at least the collected data. The individualization model is tested against quality criteria and the individualization model is approved if the quality criteria are met. The calibration period is closed if the quality criteria are met. The approved individualization model is provided providing to a system for estimating if the individual is under the influence of addictive stimulus based on eye-measurements. The approved individualization model delivers the expected values for the eye-conditions. The system for estimating if the individual is under the influence of addictive stimulus comprises a drug detection model which use a measured value and the corresponding estimated value for the purpose of determining if the measurement represents a sober condition or contains indications of drug use.

Preferably, the collecting data of each of the measurements of eye-conditions and, a respective time when the measurements of eye-conditions were performed in a handheld user interaction device also collects ambient light conditions at the time and place of measurement. Thereby, the approved individualization model delivering the expected values for the eye-conditions does so given ambient light conditions at the time and place of measurement.

In one embodiment, the calibration of the individualization model S62 for an individual is conducted using between 1 and 100 measurements, all conducted under sober conditions. More preferably, the calibration is conducted on the first about 1 to 30, or about 2 to 20, or about 3 to 10. Even more preferable is to conduct calibration on at least 5 and up to 25 sober measurements.

In one embodiment, the calibration of the individualization S62 is required to include measurements of at least two different ambient light conditions.

Preferably, the generation of the individualization model is based on at least 5 measurements of eye conditions, where the at least 5 measurements of eye conditions comprise at least one measurement made in low light ambient conditions (<100 lux), and wherein the at least 5 measurements of eye conditions comprise at least one measurement made in bright indoor light ambient conditions (>300 lux). This is particularly beneficial for detecting of class opioids or class phenetylamine.

In one embodiment, the drug detection model utilizes at least individualized data related to (a) pupil size and (b) a time-dependent aspect from the pupillary light reflex method, and where the addictive stimulus is either of class opioids or class phenetylamine.

In one embodiment, the calibration of the individualization S62 is required to include measurements of at least two distinctly different times of the day, for example “morning” and “afternoon” measurements.

In one embodiment, the calibration of the individualization S62 is required to include at least one measurement at a defined light condition at a defined time of the day. One non-limiting example could be “conduct measurement between 0800 and 1200 in 30-100 lux ambient light (which corresponds to dark conditions indoors)”.

In one embodiment, the individualization model comprises a scalar value representing the eye condition. The scalar value is estimated using either a fraction of the most recent of said at least 5 measurements or a fraction that represents the highest ability of said eye condition, so as to capture if the individual acquires or improves an ability related to the eye condition. The eye condition is here the ability of the individual to cross eyes. This is particularly beneficial for detecting drugs of class central depressant.

In some cases, it may be beneficial to bypass individualization. From a user perspective, the individualization might be perceived as a cumbersome procedure, and for some applications, individualization data may not exist. As a non-limiting example, upon starting a calibration procedure there will not be any individualization model available. As another non-limiting example, in situations where entrance control of addictive-stimulus is desired, such as the entrance to a music festival, not all attendees can be expected to have individualization models available. Still a non-limiting example is law enforcement, where law enforcement wants to estimate if an individual is under the influence of addictive-stimulus in a traffic control situation. In such a case, a global model can be provided S61 by the model selection mechanism S64 because no individualization model exists. A global model describes the population characteristics without influence of calibration measurements by the individual can be constructed. An expected value S66 would in this case refer to an expected value which is related to the population, not the individual.

The creation of a global model S61 may be different for different subpopulations. It is known that ability for eyes to adapt to light changes with age. Hence, a global model based on the individual revealing age can be constructed. From a general perspective, any readily available information about the individual which can be associated with expected change in eye characteristic can be embedded in a global model. Possible information about an individual that may impact eye characteristics and hence also a global model includes, but are not limited to, age, diabetes, and eye surgery.

As will be discussed further below, historical data of the individual can be used for revealing trend changes and may also be utilized to successively improve the baseline data.

A discussed above, ambient light may influence the measurements, and so may the situation be also for the baseline data. In preferred embodiments, the baseline data may also be characterised by an ambient light at which they are valid. In this way different sets of baseline data may be used at different occasions, depending on the surroundings of the individual when the measurements were made.

Returning to FIG. 3, in step S60, likelihood information of that the individual was exposed to an addictive stimulus is estimated, based on at least the collected data. The estimation comprises a comparison of the measurements of bodily conditions with individualized baseline measurements of the bodily conditions of the individual recorded when the individual is controlled unexposed to the addictive stimulus. In other words, the measurements are compared to a normal condition of that particular individual without drug influences. Such baseline measurements may be recorded in advance, e.g. in connection to that the procedure of using the self-administrated surveillance is agreed on. It may also be e.g. a first measurement by the user interaction device, preferably performed in the presence of a health care provider, e.g. in connection with instructing the individual about how to use the equipment. Also earlier measurements could be used for establishing a better accuracy of the individualized baseline measurements. During the first time period after the surveillance has begun, most individuals are fully motivated to be free from drugs, and such initial measurements may typically verify an accurate baseline measurement. The history of measurements is a powerful tool to distinguish untypical conditions and is preferably used for the likelihood information estimation.

Preferably, the comparison comprises a determination of an absolute or relative difference between the measurements of bodily conditions and the individualized baseline measurements and a comparison of the absolute or relative difference with a difference threshold.

In other words, the present technology presents a repeated collection of various data which is stored in a central server. The analysis of existing data during recent times forms the basis of a kind of drug sobriety index, being an individualized baseline for the measurements. At times when the drug sobriety index associated with recent measurements changes to the worse, there is a reason to reach out to the individual and for instance request a conventional drug test. This will be discussed further below.

In a preferred embodiment, the estimating of likelihood information is therefore further based on stored information about historical behaviour of the individual.

If the likelihood information exceeds a predetermined threshold, it may have been caused by a use of drugs by the individual. In such a situation, it is intended to perform step S70, in which an addictive-stimulus use-discouraging action is initiated. This addictive-stimulus use-discouraging action is by other words initiated as a response to data of the likelihood information being larger than the predetermined threshold.

The addictive-stimulus use-discouraging action can be of different kinds, or a combination of actions. A first and preferred possibility is to request the individual to perform a direct addictive-stimulus test. Such a test may the conclusively determine if drugs have been used or not. If the individual tries to hide a secret use of drugs, the “risk” of having to perform a direct test becomes discouraging, in particular if a failure to be free from drugs may have further implications, e.g. of economic or social types. Such tests may be performed by a health care provider, or if suitable self-administrated tests are available, by the individual himself/herself. If the direct tests are self-administrated, time-stamping and object-identifying functionalities are preferably to be provided.

Another addictive-stimulus use-discouraging action, possibly as a complement to the test, is to alert a pre-agreed health care provider for initiating therapy against addictive-stimulus use for the individual. Such a procedure is preferably agreed on before the surveillance procedure begins.

Another alternative or complementary addictive-stimulus use-discouraging action is to inform pre-agreed relatives of the individual about suspected addictive-stimulus use. Also this should preferably be agreed on before the surveillance procedure begins.

All such types of procedures that causes some awkward situations for the individual may put some additional pressure on the individual to in fact remain drug-free. Furthermore, if a relapse anyway occurs, the same actions can be a part of rapidly taking care of the individual.

The preferred addictive-stimulus use-discouraging action comprises requesting the individual to perform a direct addictive-stimulus test. That embodiment of the method may then be interpreted as a method for requesting a conventional drug test, preferably within its detection window.

Data collection is typically controlled through a handheld user interaction device which is capable of collecting information and connected to a central server for data deposition and preferably also capable of pushing reminders to the individual to trigger data collection. The user interaction device can preferably be a regular smartphone. When in use, the user interaction device is typically configured to request the individual to deposit data according to a pre-set schedule, e.g. at least once per day, preferably 2-4 times per day.

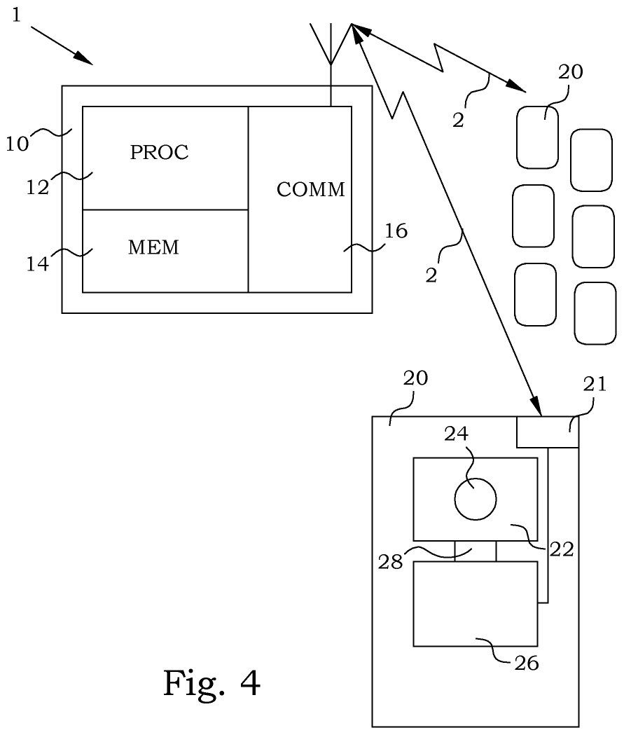

FIG. 4 illustrates schematically an embodiment of a system 1 for surveillance of use of addictive-stimulus for an individual. A central server 10 and a handheld user interaction device 20 are communicationally connected to each other, as indicated by the arrows 2. The central server 10 may be communicationally connected to a plurality of handheld user interaction devices, for different individuals. The central server 10 comprises a processor 12, a memory 14 and a communication interface 16. The central server 10, typically implemented in the processor 12, is configured for providing a body-measurement schedule for the individual to the user interaction device 20. The body-measurement schedule comprises multiple measurement time-slots. The central server 10 is further configured for requesting the individual to perform measurements of bodily conditions within each of the multiple measurement time-slots. This is made by means of the user interaction device 20, The bodily conditions comprise eye conditions. Typically, the processor 12 initiates a message to be sent via the communication interface 16 to the user interaction device 20. The user interaction device 20 comprises a communication interface for receiving the message.

The user interaction device 20 has measurement means 22, comprising a camera 24, for collecting data, and a local processor 26. The data is intended to be analysable for obtaining each of the measurements of bodily conditions. The user interaction device 20 further comprise a timer 28 for determining a respective time when the measurements of bodily conditions were performed. Typically, the processor 26 controls the measurement means 22 and the timer 28 and gathers the measurement results e.g. in the form of images and/or videos from the camera 24. The processor 26 may at least partly analyse the image/video for extracting data concerning the bodily conditions. The user interaction device 20 is further configured for transmitting the collected data from the user interaction device 20 to the central server 10 by means of the communication interface 21. The transmitted data is associated with the measurement results and may comprise the raw measurement data and/or partly or fully analysed data associated with the bodily conditions. The type and amount of data transferred to the central server is preferably adapted to the available processing power of the local processor 26 and the transmission capability between the user interaction device 20 to the central server 10. The transmitted data also comprises information about the time when the measurements actually were performed, i.e. a time stamp.

The central server 10 is further configured for receiving the collected data and storing the collected data in the memory 14. The processor 12 of the central server 10 is configured for estimating likelihood information of that the individual was exposed to an addictive stimulus, based on at least the collected data. The estimation comprises a comparison of the measurements of bodily conditions with individualized baseline measurements of the bodily conditions of the individual recorded when the individual being controlled unexposed to the addictive stimulus. Such individualized baseline measurements may be prestored in the memory 14 and/or may be successively updated by data represented drug-free measurements. The central server is further configured for initiating an addictive-stimulus use-discouraging action as a response to data of the likelihood information being larger than a predetermined threshold.

Different categories of drugs are typically detected in different ways, i.e. different indicators are searched for, for establishing a use of a particular drug. Furthermore, different drugs have different response to decomposition in the human body and give rise to different kinds of rest products. This means that different categories of drugs may have different time windows within which they can be detected. Therefore, preferably, the time for a direct test is set within a detection window, from the time of measurement, of the stimulus category to be tested.

If a general test is to be performed covering most probable stimulus categories, the shortest time window of all the drugs has to be met in order to ensure that all stimuli still are detectable.

In a preferred embodiment, the measurements can be further used also for categorizing any indicated plausible drug that has been used. To this end, the likelihood information is a set of likelihoods, one for each of a set of stimulus categories. Each likelihood is associated with a respective threshold. The direct addictive-stimulus test that will be requested after a plausible drug use has been detected is then preferably a test of the stimulus category of which the likelihood exceeds the associated threshold. In other words, the measurement analysis does not only detect possible drug use, but may also provide suggestions about which stimulus category the possibly used drug belongs to.

It is then further preferred to request the individual to perform a direct addictive-stimulus test with a set time within when the individual is to perform the direct addictive-stimulus test. This enables a more precise testing efficiency.

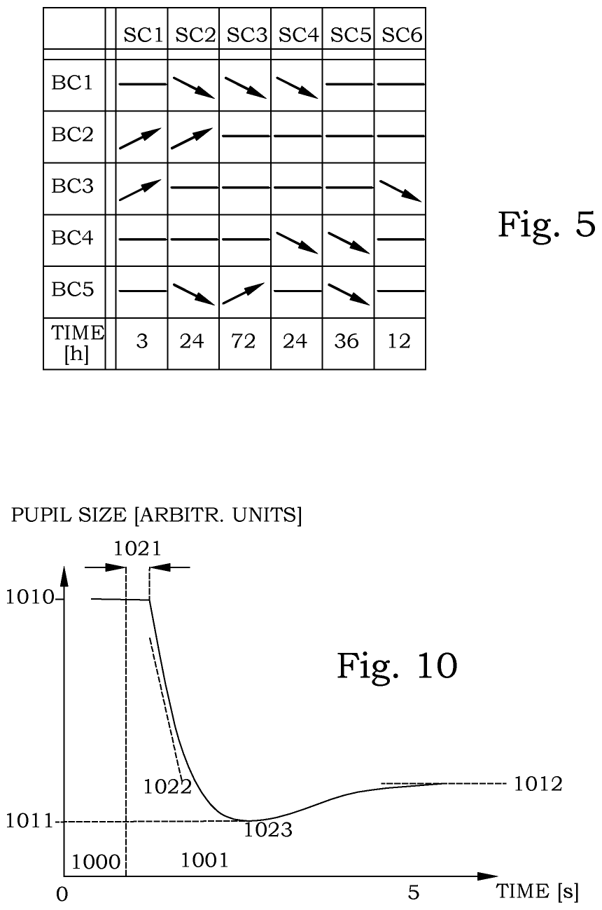

This aspect may be better understood by e.g. interpreting FIG. 5. This figure illustrates a table of six different stimulus categories SC1-SC6 and five different bodily conditions BC1-BC5. For drug category SC1, investigations may have shown that use of a drug of stimulus category SC1 has a tendency to increase the measurement results associated with bodily condition BC2 and BC3, while measurement results associated with bodily conditions BC1, BC4 and BC5 are essentially unaffected by drugs of stimulus category SC1. Likewise, stimulus category SC2 is associated with a decrease of BC1 and BC5, an increase of BC2 and no effect on BC3 and BC4. Stimulus SC3 is associated with a decrease of BC1 and an increase of BC5 and no effect on BC2-4. Stimulus SC4 is associated with a decrease of BC1 and BC4 but no effect on BC2, BC3 or BC5. Stimulus SC5 is associated with a decrease of BC4 and BC5 but no effect on BC1, BC1 or BC3. Stimulus SC6 is associated with a decrease of BC3 but no effect on BC1, BC2, BC4 or BC5.

By matching the obtained measurements to such a table, there are increased possibilities to discriminate a use of a drug of one of the stimulus categories from other stimulus categories. Furthermore, since associated direct tests may be different for the different stimulus categories and/or the test window within which the direct test has to be performed may be different, a more appropriate choice of test and test time can be found.

In FIG. 5, test window times are indicated at the bottom of the table. The stimulus category SC1 test window is short, just 3 hours. Therefore, it might be difficult to practically arrange for such a test. However, if the bodily condition measurements are such that they indicate that the likelihood for drugs of stimulus category SC1 is low, appropriate tests can be requested having much longer test windows. Thereby, both the accuracy and the practical efforts of the direct tests can be improved.

A table like the one of FIG. 5 can be constructed for the drugs of interest and for the available bodily condition measurements. It is for instance typically considered that central depressive substances make pupil light reaction slower and also affects nonconvergence as well as horizontal and vertical nystagmus. At the contrary, use of cannabinoids are believed to result in normal or enlarged pupil size and effect son nonconvergence, but not affecting pupil light reactions or nystagmus. Central stimulants are believed to result in enlarged pupil size and slower pupil light reactions. Opioids instead give reduced pupil sizes and no or minor reaction concerning pupil light reactions.

The selection of stimulus categories and measured bodily conditions is preferably determined by the scope of the surveillance and of which measurements that are available, as well as of where there is reliable scientific proof of their connections.

An important difference between monitoring drug addicts and alcohol addicts is that for alcohol, the self-administrated measurement may be direct and confirmatory in the sense that the alcohol is measured directly in a device such as a breathalyzer. At a contrary, according to the present ideas, measurements for drug addicts, other than alcohol, only are indicative. Pupil reaction changes can occur because of widely different causes, e.g. drug use, falling in love, being extremely tired, and so on. The advantage of an indirect measurement is however the broad scope. Synthetic designer drugs, like chemical derivatives of amphetamine that affect the body in about the same manner but that are difficult to measure directly because the chemical structure is novel for each designed case, will be easier to detect using indirect methods. The error rate for an indirect method will, however, be higher, because other unrelated states may result in the same bodily reaction as drug use. Therefore, a confirmatory direct test made on a body fluid (for example breath, saliva, urine, or blood) is to prefer in addition.

Alcohol addiction monitoring concepts have taught that the value of a single test result is moderate, even when direct measurements are used, basically due to a relatively short test window for alcohol. Instead, improvements were shown when results from different sources, of which direct measurements is one, were combined, preferably also monitored over time. A reliable continuous picture of the health status of the individual can be obtained in such a way. An indirect method like pupillometry is found to be adequate and good-enough for the purpose of following a drug use disease state over time, only supported by occasional confirmatory direct tests.

Changes in measurement trends may therefore be a complement to the measurements themselves. A small but distinct change in a general trend may be as indicative as a single measurement indicating a clear possible drug use. Also, for measurements having large uncertainties, the levels for indicating a suspected drug use may be set very conservative in order to avoid too many false detections. However, if such relatively uncertain measurements are repeated relatively frequently, the statistics will improve the overall precision of the determinations of likelihoods.

Collected data need to be combined over time so as to depict the status of the individual in the light of recently collected data.

Visual conditions of eyes, i.e. pupillometric data, have been identified as a useful source of information. A device can be configured to collect video films of the eyes of the subject. Such a collection is furthermore easily managed by the subject himself/herself. Thereby, multiple different characteristics can be extracted. The pupil response to light changes. The ability of the subject to cross his/her eyes can be followed. Voluntary and unvoluntary eye movement patterns, i.e. nystagmus/saccadic aspects can be followed. The colour of the whites of the eye can be determined, etc. Many of these are known to be more or less capable of indicating use of drugs. Each characteristic is, however, typically linked in different ways to different stimulus categories, as mentioned above. Preferably, the eye conditions are selected from pupil size, pupillary light reflex, behaviour of crossing eyes, nystagmus, saccadic eye movements and colour of eye whites.

However, it has been found that pupillometric data may be complemented also by other types of data in order to further increase the detectability of drug use. Other bodily conditions may e.g. be measured. In one embodiment, bodily conditions therefore also comprise motion conditions. The device can be configured to collect data related to how the individual handles the device, for example using an accelerometer in the device or using image analysis to investigate blurring or moving of a video-stream captured by the device. Thus, in one embodiment, the motion condition is accelerometer measurements of hand motion and/or stability of camera by analysing collected video filmed by the individual.

In one preferred embodiment, the bodily conditions are at least two different bodily conditions. These two different bodily conditions may be two eye-related conditions, e.g. pupil size and ability to cross eyes. The two different bodily conditions may e.g. also be one eye related and one motion condition, such as e.g. pupil size and accelerometer measurements. In a simplified general view, the more types of bodily conditions that can be recorded, the more accurate the likelihood measurements may be.

The collected data may also comprise other data, not directly connected to bodily conditions. Useful elements in data collection, supporting the bodily condition measurements, may e.g. be questionnaires to reply on. The device can for instance be configured to ask the individual a number of questions, for example questions related to motivation, mood, and well-being. Inability to conduct a requested test, i.e. when an individual, when requested, tries to conduct a test but fails may also be of interest. Furthermore, a confession from the individual that drugs indeed have been used in recent times is of course of benefit for interpreting the total situation. Also the existence of an intentional or unintentional omission of a requested test can be used during the analysis.

In one non-limiting example, the use of addictive-stimulus of class “central depressants” is visible in the eye function of most individuals. The class of central depressants include, but are not limited to, benzodiazepines, ethanol (alcohol), barbiturates, methaqualone, and gamma hydroxybutyrate (GHB). One eye function that is impaired upon ingesting a sufficiently high dose of central depressants is the ability to cross eyes (also known as non-convergence). However, a small percentage of the population does not have the ability to cross eyes [The Rapid Eye Test to Detect Drug Abuse August 1988, Postgraduate Medicine 84(1):108-14, DOI:10.1080/00325481.1988. 11700339]. The ability to, and the magnitude of, crossing eyes is further an acquired skill, i.e. an individual becomes better at crossing eyes the more he/she practices. Hence, in order to utilize a measurement of the ability of an individual to cross eyes for the purpose of detecting drugs, an individualization capable of managing both lack of ability as well as increasing ability to cross eyes is required. The individualization model for the ability to cross eyes could be a scalar value which is representative of an individual's capacity to cross eyes.

In one embodiment, the calibration of individualization S62 (FIG. 6) is sufficiently long and adapted to allow calibration of an acquired skill, such as for example the skill of crossing eyes. Such a calibration procedure could for example rely on a fraction (such as 30% or 50%) of the most recent measurements, hence disregarding the initial attempts that could be of underperforming nature if the skill was not yet acquired. As another example, such a calibration procedure could rely on a fraction (such as 30% or 50%) of the strongest results indicating the acquired skill, hence disregarding any attempts that could be of underperforming nature irrespective when in time they may have occurred.

In one embodiment, the individualization includes an adaptive element S76 (FIG. 6) that updates the individualization so as to allow continuous calibration of an acquired skill. A non-limiting example includes the skill of crossing eyes.

In one embodiment, a drug detection model which use the acquired skill of crossing eyes for the purpose of detecting the addictive-stimulus of class central depressants, such as benzodiazepins is provided. This drug detection model would need an individualization model that can manage the acquired skill.

Collected data is preferably combined into information-rich features to make evaluation easier. For example, data from a clinical trial can be subjected to principal component analysis to elucidate which combinations of collected data that most efficiently distinguish drug use. As one example, use of cannabis is known to induce red eyes and is known to dilate the pupils. By creating a cannabis specific feature that combines the colour of the whites of the eye and the pupil size, a more specific combinatorial indicator for cannabis use is created. Such a specific combinatorial indicator will to a lesser degree indicate suspected cannabis use if an individual gets red eyes from slicing onions in the kitchen.

Another example where combinations of collected data efficiently distinguishes addictive-stimulus use relates to opioids. The drug class opioids include, but is not limited to, morphine (naturally occurring), heroin (semisynthetic), meperidine and methadone (synthetic derivatives) and prescription opioids including tramadol, hydrocodone, oxycodone, pentazocine and fentanyl. It is well known that use of opioids causes miosis, i.e. small pupils. It is also known that bright light causes pupils to shrink to a small size. At low light conditions, the small pupil originating from opioid use would be clearly distinguishable from the normal pupil size. However, there is a challenge in separating small pupils originating from use of opioids and from bright ambient light. The challenge is two-fold: An individualization, if implemented, need to function at all acceptable ambient light conditions, and the addictive stimulus identification model need to function at all acceptable ambient light conditions. In such a bright light situation a measurement of the pupillary light reflex can support distinguishing the cause of the small pupil. A pupillary light reflex measurement can be conducted by video-filming the eyes of an individual before, during and after a short light pulse. As a non-limiting example, a smartphone can be programmed to collect a video sequence during which the flashlight of the smartphone is activated a few seconds. When extracting pupil size for each frame in the video, compiling a pupillogram (pupil size over time), and calculating different characteristics of the entire pupillogram, use of opioids can be distinguished from small pupils caused by only bright light through combining the different characteristics. For example, the individual using opioids may have less pupil size reaction from activating the flashlight as well as smaller recovery of pupil size after illumination is stopped. By combining characteristics like “pupil size before measurement”, “pupil size when contracted”, and “contraction velocity”, characteristics of the eye can distinguish use of opioids from sober condition also at bright ambient light.

In one embodiment, the addictive-stimulus model S67 (FIG. 6) is designed to detect addictive-stimulus of the class opioids using data from a pupillary light reflex measurement and combining (a) pupil size and (b) time-dependent aspect from the pupillary light reflex method, all subjected to an individualization model that relies on ambient light conditions at the time and place of measurement, into a single composite value. Pupil size could for example be represented by “pupil size before measurement”, “pupil size when contracted”, or similar. A time dependent aspect from the pupillary light reflex method could for example be “contraction velocity”. The single composite value is, when above or below a predefined threshold value, indicative of use of opioids.

In one embodiment, the addictive-stimulus model S67 (FIG. 6) is designed to detect addictive-stimulus of the class phenetylamines using data from a pupillary light reflex measurement and combining (a) pupil size and (b) time-dependent aspect from the pupillary light reflex method, all subjected to an individualization model that relies on ambient light conditions at the time and place of measurement, into a single composite value. The class phenetylamines includes, but is not limited to, cocaine, amphetamine, methamphetamine, and 3,4-methylenedioxymethamphetamine (MDMA, ecstasy), cyclazodone, 4-methylaminorex, lisdexamphetamine, methylphenidate, pseudoephedrine, phenylephrine, promethazine, phenylpropanolamine and oxymetazoline.

Pupil size could for example be represented by “pupil size before illumination”, “pupil size when contracted”, or similar. A time dependent aspect from the pupillary light reflex method could for example be “time to maximum contraction” into a single composite value. The single composite value is, when above or below a predefined threshold value, indicative of use of phenetylamines.

A preferred key feature of the present invention is the ability to estimate the likelihood of an individual using addictive-stimulus based on historic data, gradually available in the central server. In addition to indirect actual measurements of addictive-stimulus (for example evaluating the pupillary response to light), answers to questionnaires, and missed tests, the behavioral pattern in how scheduled tests are actually performed may correlate to risk for having used addictive-stimulus. The behavioral pattern may be related e.g. to compliance to the scheduled body-measurement tests, i.e. that the individual actually performs the tests as scheduled, but also to the manner in which the tests are performed, even when within requested scheduling. By monitoring behavioural patterns, attempts to manipulate become visible and are useful indicators in the evaluation of risk for having used addictive-stimulus. An individual who at a certain point in time becomes at elevated probability for having used addictive-stimulus may furthermore intentionally attempt to adjust the scheduling so that longer periods of time are unmonitored. For example, by claiming morning stress and requesting the schedule to move the morning exposure test forward in time a seemingly short time, such as for example 0.25, 0.5, 0.75, 1, 1.5, 2, 2.5, 3, 3.5 or 4 hours or anything similar, could be an indication.

As a non-limiting example, one simple yet potentially powerful behavioral indicator is “time between body-measurement tests”, which is the amount of time that has passed since the most recent accepted body-measurement test. A day can be represented by the “maximum time between body-measurement tests” (MTBBMT), which is the maximum time between body-measurement tests of all available time between exposure tests in a given day. Another non-limiting example is change in the average total time required to complete body-measurement test.

It is possible and desirable to use many different underlying methods for determining if the addictive-stimuli exposure history, e.g. intoxication history, and body-measurement test behavioural patterns are indicative of an individual being clean. One non-limiting method is to make a weighted average of the measurement results related to suspected addictive-stimuli exposure the recent year, where newer measurements are given higher weight than older. Should an individual be confirmed exposed to addictive-stimuli, for example through a direct test or through confession, at one point in time, this exposure event will have high impact on the soberness estimation shortly after, but will gradually reduce in impact as time passes. Another possible method is to extract a short-term suspected addictive-stimuli use history which relates e.g. to the previous month and a long-term suspected addictive-stimuli use history that relates e.g. to approximately the recent year or years, and evaluate if both the short term and the long term history are indicative of suspected use of addictive-stimuli. In the short term assessment, the onset of a problematic period can be captured, while as in the long term assessment, the change of the suspected addictive-stimuli use habit of the individual can be monitored. This makes it possible to determine if a present suspected addictive-stimuli use event is likely an accident, if the long term history suggests that the individual is staying sober most of the time, or if it likely is part of a pattern suggesting a larger decay into use of addictive-stimuli. Such a procedure can be discussed in more detail in the non-limiting context of use of intoxicating chemicals such as cannabis. One possible non-limiting example of a method for combining short term intoxication history with medium term or long term intoxication history for the purpose of determining the cleanness of an individual is the following. First, a short term intoxication (STI) value is calculated. This STI value can be constructed in many different ways, but one possible definition is the following:

$$STI = ( ( mild suspected intoxication days the recent 30 days ) + 3 * ( severe suspected intoxication days the recent 30 days ) + ( average MTBBMT the recent 30 days ) ) / 30$$

The expression “mild intoxication day” could e.g. refer to a day when a body-measurement produces a value above but near the value expected in sober conditions is registered. “Severe intoxication day” could e.g. refer to a day when a body-measurement produces a value which is much higher value expected in sober conditions is registered.

Second, a medium term intoxication (MTI) value can be calculated in a similar manner. One possible definition is the following:

$$MTI = ( STI [ 30 ] * 3 + STI [ 60 ] * 2 + STI [ 90 ] * 1 ) / 6$$

wherein STI[x] represents the historic STI value at x days before the day of calculating the MTI value, so that STI[1] represents the STI value of yesterday, and STI[2] represents the STI value of the day before yesterday. In plain words, MTI could e.g. be defined as the weighted sum of three historic STI values (in this example one month ago, two months ago and three months ago) where the most recent historic STI value is given higher weight and the oldest STI value is given lowest weight. A long term intoxication (LTI) value can be calculated in still a similar manner, but covering a time period greater than the MTI value (>3 months in this example). Similar short-term, medium term and long term indicators can be designed also for other stimuli than intoxicating compounds.

Motion conditions may be a body-measurement data which is useful for suggesting use of addictive stimuli. It is possible to quantify motion conditions in many ways, and one non-limiting example is to denote a variable QM for the evaluation of the amplitude of tremor of the limb which holds the measurement device in the frequency span 1-10 Hz.

By combining intoxication measures of different time-frames like e.g. STI, MTI, LTI, QM, MTBBTT and/or other data a reliable measure of the likelihood of suspected use of addictive-stimuli can be created. Other data includes, but is not limited to, time of day, day of week, type of day (e.g. holiday, salary day etc.), result of the previous measurement, elapsed time since the previous measurement, number of measurements that have been missed in the recent past, and similar. It is important to note that since many pieces of information are combined into a joint probability for suspected use of addictive-stimuli, the quality requirement on each contributing factor is moderate. This is because it is sufficient that any one of the conducted tests or tasks is suggesting use of addictive-stimuli for the average probability to increase and potentially be considered reportable.

In some cases it may, for pedagogic reasons in relation an individual suffering from addictive disorder, be favorable to present an intoxication index as a sobriety index. A sobriety index is always negatively correlated with an intoxication index.

The reaction of pupils to light and stimuli is dependent on ambient light conditions. This has been discussed e.g. by Ong and co-authors in “The Effect of Ambient Light Conditions on Quantitative Pupillometry”, in Neurocrit Care (2019) 30:316-321. For example, in bright ambient light, the pupillary light reflex is smaller in magnitude, because the baseline pupil size is smaller due to the ambient light.

Therefore, preferably, the measurements may also be related to a function of ambient light.

The presented technology provides a method for continuously estimating if an individual has signs of drug use, and hence would be in need of a conventional drug test. When using the method of this technology, the possibility for the individual to hide drug use through planned use may be eliminated, because the individual would expose him/herself to an indirect test that with high probability would suggest that drugs have been consumed when the individual indeed has consumed drugs. Upon noting elevated probability of drug use, a health care provider can choose to act. Examples of suitable actions include, but are not limited to, provide support or therapy to the individual, contact the family of the individual, or request a conventional drug test. In the case of requesting a conventional drug test, it would with high likelihood be placed within the test's detection window.

### Example 1

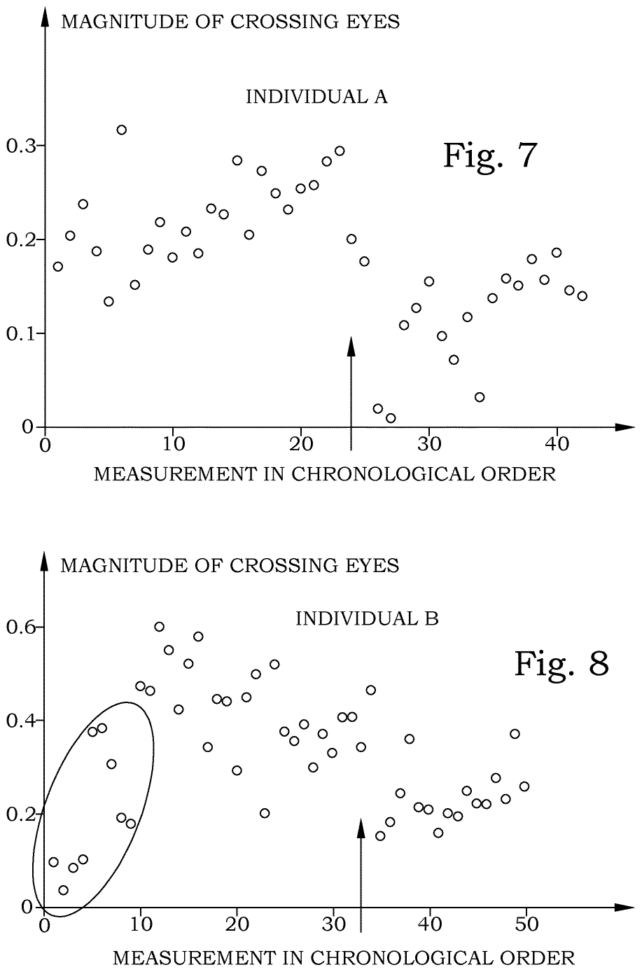

The following example relies on data from the clinical study KCClin01, a clinical study registered at ClinicalTrials.gov with identifier NCT05731999. In this clinical study, healthy volunteers (HV) were subjected to self-administered eye measurements using a smartphone. To begin with, each HV conducted eye measurements in sober condition both at the study center and at home. Later, each HV was subjected to selected drugs at the study center under strict supervision of clinical staff. FIG. 7 shows results for individual A and the eye measurement related to ability to cross eyes. The value indicating an ability to cross eyes describes is always between 0 and 1, where 0 represents looking straight and 1 represents turning each eye approximately 90 degrees towards the nose. Individual A was given a drug of class benzodiazepine between measurement 23 and 24, indicated with an arrow. Individual A had the ability to cross eyes prior to embarking in the clinical study, and the ability to cross eyes was partially impaired by administration of a benzodiazepine drug. FIG. 8 shows results for individual B and the eye measurement related to ability to cross eyes. Individual B was given a drug of class benzodiazepine between measurement 32 and 33, indicated with an arrow. Individual B had difficulties to cross eyes prior to embarking in the clinical study but learned to do so during the initial about 10 measurements, as indicated by encircled data. The ability to cross eyes was partially impaired by administration of a benzodiazepine drug when comparing to the acquired skill of crossing eyes (measurement numbers 10-30) but not when compared to the initial phase of measurements during which individual B was still learning to cross eyes.

When comparing Individual A and B, clear individual differences are seen. Individual A manages to cross eyes with a magnitude of approximately 0.2 in sober conditions, whereas individual B managed, after some training, to cross eyes with a magnitude of approximately 0.4. The typical sober condition crossing eye magnitude of Individual A represents an impaired crossing eye ability of Individual B (after training). This means that interpretation of the ability to cross eyes for the purpose of detecting drugs will require an individualization (because baseline sober ability differs widely) which is capable of adapting to acquired skills (because some individuals change their sober ability through training).

### Example 2

The following example relies on data from the clinical study KCClin01, a clinical study registered at ClinicalTrials.gov with identifier NCT05731999. Data from the first third of the study was used to produce this example, comprising 1278 measurements made by 17 individuals from KCClin01 and 15 other HV that contributed with sober data only.

In the study protocol of KCClin01, HV were requested to make eye-scanning measurements at home three times per day one week before the study visit where they were given a drug. This resulted in approximately 20 sober data points. Data from at home was considered as a source for “sober calibration data”. An individualization model was fitted using 10 data points of sober calibration data. In this case, the individualization model was the estimated ability, for an individual, to cross eyes after 10 ten attempts calculated as a weighted average where results from later attempts were given higher weight than results from earlier attempts.

Now, the individualization model was applied on the remaining about 10 data points. The value “deviation from own expected value” (DFOEV) was calculated in the following manner:

$$DFOEV = MeasurementValue - IndividualizationModelOutput$$

Where MeasurementValue is the value measured in the eye scanning process, and IndividualizationModelOutput is the value produced by the individualization model for the current measurement.

At this stage, for each HV there was about 10 data points expressed as DFOEV. Then, the process continued to evaluate also measurements made under the influence of drugs.

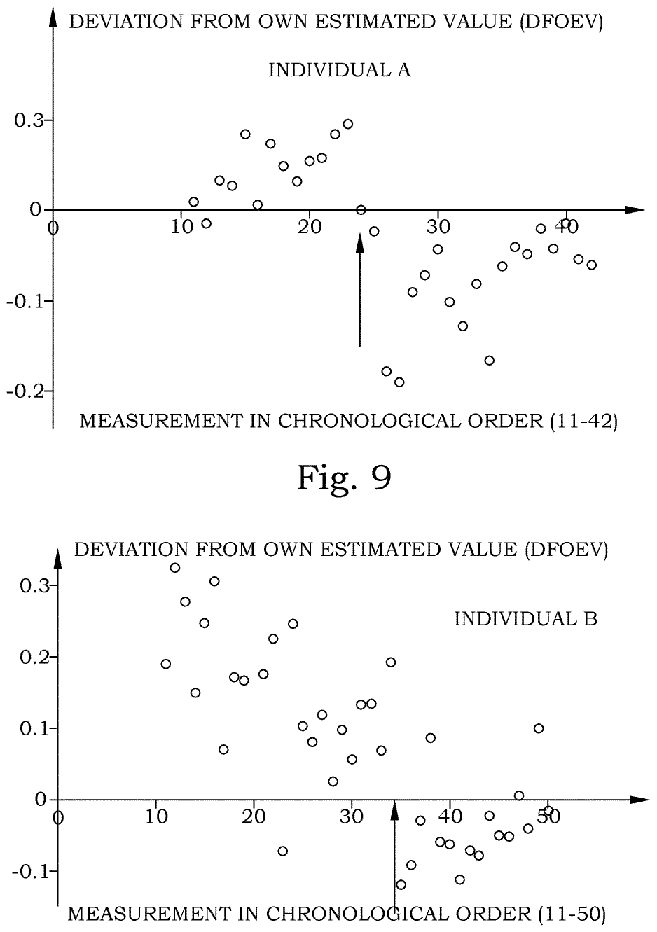

In this example, two HV (same two as in Example 1) were evaluated. These two HV were provided drugs of the class benzodiazepines. DFOEV values for measurement occasions 11 and upwards are shown in FIG. 9. Drug onset is shown as an arrow in each graph. FIG. 9 demonstrates that very different raw data (FIG. 7, FIG. 8) can be transformed into values that are easier to compare. In this particular case, a DFOEV which is smaller than 0 is highly indicative of benzodiazepine use, irrespective of the original ability of crossing eyes, and if the ability of crossing eyes was acquired during the course of calibrating the individualization model.

Hence, this example shows that individualization of data is possible and may be necessary to enable the detection of drugs in eye-scanning results.

### Example 3

The following example relies on data from the clinical study KCClin01, a clinical study registered at ClinicalTrials.gov with identifier NCT05731999. Data from the first third of the study was used to produce this example, as previously described.

In this example, the ability to detect phenetylamines is described. Among the available data for this example, three HV were provided “lisdexamphetamine”, which is a member of the phenetylamine family. Phenetylamines are known to widen the pupils, irrespective of surrounding light.

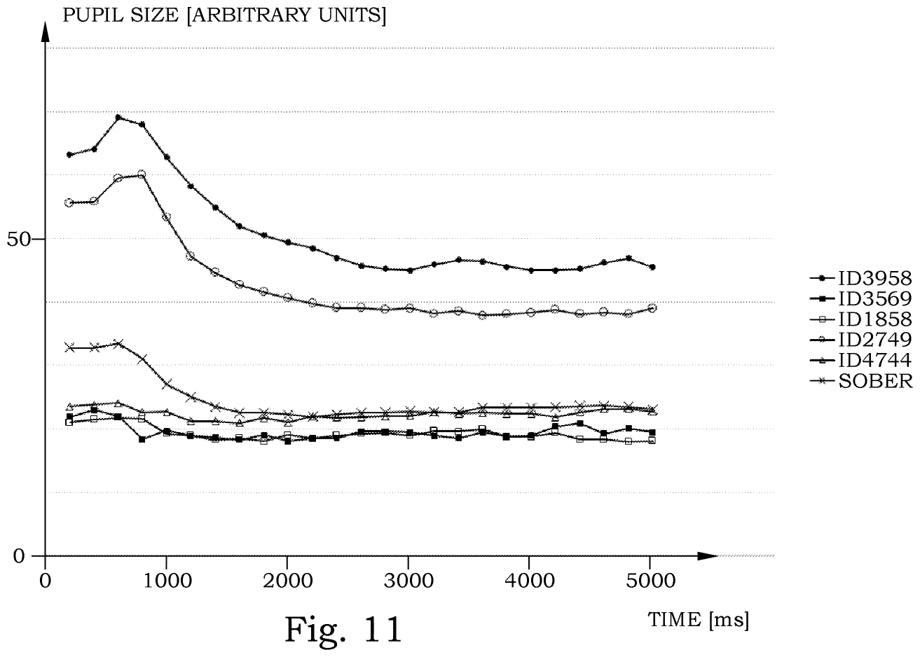

Data from the pupillary light reflex measurement were extracted for this example. FIG. 10 describes a schematical and typical pupillary light reflex data where the pupil size is depicted over time. Illumination is first off 1000 and shortly after starting measurement turned on 1001 for the duration of about 5 seconds. At first, the pupil size is at Dbase 1010 level. There is a reaction time, often denoted latency 1021, from the time of activating the illumination to first visible reaction of the eye. Next there is a quick reaction of the eye in reducing the pupil size, where a maximum contraction velocity (MCV) 1022 can be estimated. At contraction time (Ctime) 1023 the pupil has reached the smallest size Dcon 1011. The pupil then increases slightly to reach a final size Dend 1012.

In a first attempt, raw pupil size measurements prior to activating light (Raw Dbase) were used to distinguish data related to sober HV from data related to HV under the influence of a phenetylamine (Table 1, row “Raw Dbase”). This led to a statistically significant difference between the conditions. However, the separation of the averages is small, where the differences in average of sober data and phenetylamine data are only 0.15 standard deviations (for sober data) apart. This means that, assuming normal distribution, approximately 45% of the sober Raw Dbase data was larger than the phenetylamine average Dbase. This means that the significant difference between the sober and phenetylamine condition is meaningless from a diagnostics point of view, because too many false positive results would be generated.

Next, a global model for Dbase was taken into consideration. The global model accounts for Dbase at different ambient light levels, and also for the age of the HV. In practice, the population Dbase average at different age groups (20-30 years, 31-50 years, older than 50) at a few light intensities were estimated from available data. Thereafter, an expected value could be generated by first providing the ambient light level and age of the HV, and then interpolating the expected value from the table with population Dbase averages. The output for Dbase alone after applying the global model is shown in table 1, row Global Dbase. The absolute values differ from the Raw Dbase case, because now the unit is “deviation from expected value” (DFOEV). The sober DFOEV is close to 0, which means that the global model is indeed depicting the sober behaviour for Dbase. When comparing average sober DFOEV and phenetylamine DFOEV, the difference is 1.83 standard deviations (of sober DFOEV) which means that, assuming normal distribution, approximately 3.5% of the sober DFOEV data was larger than the phenetylamine average DFOEV. Every 29^{th} sober measurement hence produced a larger value than the phenetylamine average, which is a great improvement compared to Raw Dbase.

Next, the global model was extended to several key features from a pupillary light reflex measurement, namely (1) time to maximum contraction (Ctime), pupil size at maximum contraction (Dcon), pupil size at end of measurement (Dend) and Dbase as defined above. The global model accounts for each key feature value in the same manner as described above, with the exception that age was not used for stratification for Dend and Ctime. It is noted that Dbase, Dcon and Dend relate to pupil size magnitude, whereas Ctime relates to the time it takes for the eye to react to light. A composite value was defined, comprising the sum of all key feature values. The output for the composite value is in DFOEV units and is shown in table 1, row Global multi. The sober multi DFOEV is close to 0, which means that the global model as applied in a combinatorial manner is indeed depicting the sober behaviour for the composite value. When comparing average sober multi DFOEV and phenetylamine multi DFOEV, the difference is 2.0 standard deviations (of sober multi DFOEV) which means that, assuming normal distribution, approximately 2.5% of the sober multi DFOEV data was larger than the phenetylamine average multi DFOEV. Every 40^{th} sober measurement hence produced a larger value than the phenetylamine average, which is an improvement compared to Global Dbase.

This example hence shows that a global model can make a drug test functional, and that a combinatorial approach where several key features are combined, preferably of different categories (magnitude, time or velocity, etc) is beneficial.

TABLE 1
Determinations of the influence of a phenetylamine.

| | | Sober | | | |
|---|---|---|---|---|---|
| | Sober | standard | Phenetylamine | | % False |
| Condition | average | deviation | average | Separation | positive |
| Raw | 38.3 | 9.1 | 39.6 | 0.15 | ~45% |
| Dbase | | | | | |
| Global | 0.05 | 0.95 | 1.79 | 1.83 | ~3.5% |
| Dbase | | | | | |
| Global | 0.05 | 1.56 | 3.18 | 2.0 | ~2.5% |
| multi | | | | | |

### Example 4

The following example relies on data from the clinical study KCClin01, a clinical study registered at ClinicalTrials.gov with identifier NCT05731999. Data from the first third of the study was used to produce this example, as previously described.

In this example, the ability to detect opioids is described. Among the available data for this example, three HV were provided “oxycodone”, which is a pharmaceutical that is a member of the opioid family. Opioids are known to shrink the pupils, irrespective of surrounding light.

Similar to example 3, Raw Dbase from the pupillary light reflex measurement were used to distinguish data related to sober HV from data related to HV under the influence of an opioid (Table 2, row “Raw Dbase”). This led to a statistically significant difference between the conditions, where the separation was 1.4 standard deviations (corresponding to ˜8% of sober data being smaller than the sober Dbase average).

Next, a global model for Dbase was taken into consideration. The output for Dbase alone after applying the global model is shown in table 1, row Global Dbase. The absolute values differ from the Raw Dbase case, because now the unit is “deviation from expected value” (DFOEV). The sober DFOEV is close to 0, which means that the global model is indeed depicting the sober behaviour for Dbase. When comparing average sober DFOEV and opioid DFOEV, the difference is 2.63 standard deviations (of sober DFOEV) which means that, assuming normal distribution, approximately 0.43% of the sober DFOEV data produced a larger value than the opioid average DFOEV.

Next, the global model was extended to several key features from a pupillary light reflex measurement, namely (1) time to maximum contraction velocity (MCV), pupil size at maximum contraction (Dcon), pupil size at end of measurement (Dend) and Dbase as defined above. The global model accounts for each key feature value in the same manner as described above, with the exception that age was not used for stratification for Dend and MCV. It is noted that Dbase, Dcon and Dend relate to pupil size magnitude, whereas MCV relates to the velocity of the eye reaction to light. A composite value was defined, comprising the negated sum of all key feature values. The output for the composite value is in DFOEV units and is shown in table 2, row Global multi. The sober multi DFOEV is close to 0, which means that the global model as applied in a combinatorial manner is indeed depicting the sober behaviour for the composite value. When comparing average sober multi DFOEV and opioids multi DFOEV, the difference is 2.66 standard deviations (of sober multi DFOEV) which means that, assuming normal distribution, approximately 0.39% of the sober multi DFOEV data was larger than the opioid average multi DFOEV. Every 256^{th} sober measurement hence produced a larger value than the opioid average value.

Finally, an individualized model was constructed for the key features used in the global multi case. The individualized model was similar to the global multi model (described in example 3 and above) with the distinct difference that a calibration had been made so as to provide values from the individual into the model, replacing population values. In practice, a combination of the individual average of any key feature at a few light intensities (as estimated from available data originating from the individual) and the corresponding values from the global model were used as basis for individualization. To rely in part on the global model means that also a few measurements from the individual are sufficient to tweak the model from the global values towards the individual values, without requiring a full set of individual values that populate the entire space of light intensities. A composite value was defined, comprising the negated sum of all individualized key feature values. The output for the composite value is in DFOEV units and is shown in table 2, row Individualized multi. The sober individualized DFOEV is close to 0, which means that the global model as applied in a combinatorial manner is indeed depicting the sober behaviour for the composite value. When comparing average sober individualized DFOEV and opioids individualized DFOEV, the difference is 2.98 standard deviations (of sober individualized DFOEV) which means that, assuming normal distribution, approximately 0.14% of the sober multi DFOEV data was larger than the opioid average multi DFOEV. Every 694^{th} sober measurement hence produced a larger value than the opioid individualized DFOEV average.

TABLE 2
Determinations of the influence of oxycodone.

| | | Sober | | | |
|---|---|---|---|---|---|
| | Sober | standard | Opioid | | % False |
| Condition | average | deviation | average | Separation | positive |
| Raw Dbase | 38.3 | 9.11 | 25.2 | 1.4 | ~8% |
| Global Dbase | −0.05 | 0.95 | 2.46 | 2.63 | ~0.43% |
| Global multi | −0.12 | 1.65 | 4.3 | 2.66 | ~0.39% |
| Individualized | −0.13 | 1.71 | 4.96 | 2.98 | ~0.14% |
| multi | | | | | |

This example hence shows that an individualized model can contribute to elevated performance of a drug test, and that a combinatorial approach where several key features are combined, preferably of different categories (magnitude, time or velocity, etc) is beneficial.

### Example 5

The following example relies on data from the clinical study KCClin01, a clinical study registered at ClinicalTrials.gov with identifier NCT05731999. Data from the first third of the study was used to produce this example, as previously described.

In previous examples, separation between a sober value and a value obtained under the influence of a defined drug has been discussed in units of the standard deviation of sober values. It is evident in the data from KCClin01 that the standard deviation in key feature values is greater in the afternoon compared to the morning. This may be due to the eyes have been exposed to a variety of ambient light situations during the day, leading to larger variability in the afternoon. Table 3 shows the standard deviation in a cohort of sober HV for the maximum contraction amplitude (MCA) expressed as individualized DFOEV at different ambient light levels and at different times of the day. Similar results were obtained for all key features from pupillary light reflex measurements (FIG. 10).

TABLE 3
standard deviation for the maximum contraction amplitude
expressed as individualized DFOEV at different ambient
light levels and at different times of the day.
Standard deviation for individualized MCA results

| | |
|---|---|
| Time of | Ambient light ranges |

| day ranges | 50-155 lux | 156-500 lux |
|---|---|---|
| 0900-1200 | 2.39 | 2.31 |
| 1201-1700 | 2.66 | 2.19 |

This example hence shows that a global model or an individualization model may benefit from use of a time-of-day dependent representation of the key feature standard deviation for the sober population.

### Example 6

The following example relies on data from the clinical study KCClin02, a clinical study registered at ClinicalTrials.gov with identifier NCT05737550. In this study, a number of individuals who were confirmed patients with substance use disorder conducted self-administered eye-scanning tests. Data related to pupil size, pupillary light reflex, behaviour of crossing eyes, nystagmus, saccadic eye movements and colour of eye whites were collected. The intoxication status of each individual was determined through a combination of time-line follow-back (i.e. a questionnaire) findings and analysis of drug residues in breath samples using the Breath Explore device. In average, each individual in the clinical study had ingested more than 2 different drugs during the recent 24 h when conducting a self-administered eye-scanning test. Data from pupillary light reflex measurements presented in FIG. 11 reveal that an individual recently ingesting heroin (ID1858) and another individual recently ingesting tramadol (ID3569) resulted in reduced pupil size. Also, an individual recently ingesting amphetamine (ID3958) and another individual ingesting MDMA “crystal meth” (ID2749) resulted in enlarged pupil size. As a comparison, data from an individual outside the KCClin02 study is also presented in FIG. 11. This individual made several measurements in sober conditions (one of these shown as “Sober” in FIG. 11) and later underwent a medical procedure where 50 mcg fentanyl (an opioid) was prescribed and administered. Data collected 1 h after fentanyl administration is shown as ID4744.

Hence, the present invention is not limited to any particular drug substance, but rather to a group of substances belonging to a particular family, where any member of a family has similar implications on the physiology of the individual who ingests the drug.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1. A method for self-administrated surveillance of use of addictive-stimulus for an individual, comprising the steps of:
providing a body-measurement schedule for said individual, said body-measurement schedule comprising multiple measurement time-slots;
requesting said individual to perform measurements of bodily conditions within each of said multiple measurement time-slots;
said bodily conditions comprise eye conditions analysable from camera recordings;
collecting data of each of said measurements of bodily conditions and a respective time when said measurements of bodily conditions were performed in a handheld user interaction device;
transmitting said collected data from said user interaction device to a central server;
storing said collected data in said central server;
generating individualized baseline data for an individualization model associated with an individual, said individualization model comprising data of eye conditions analysable from camera recordings when being unexposed to any addictive stimulus;
said generation of said individualization model being based on at least 5 measurements of eye conditions when being unexposed to any addictive stimulus;
estimating likelihood information of that said individual was exposed to an addictive stimulus, based on at least said collected data;
said estimating comprises comparison of said measurements of bodily conditions with said individualized baseline data associated with said individual, said individualized base line data comprising data of said bodily conditions when being unexposed to said addictive stimulus; and
initiating an addictive-stimulus use-discouraging action as a response to data of said likelihood information being larger than a predetermined threshold.

2. The method according to claim 1, wherein said addictive-stimulus use-discouraging action is selected as at least one of:
requesting said individual to perform a direct addictive-stimulus test;
alerting a pre-agreed health care provider for initiating therapy against addictive-stimulus use for said individual; and
informing pre-agreed relatives of said individual about suspected addictive-stimulus use.

3. The method according to claim 2, wherein said addictive-stimulus use-discouraging action comprises requesting said individual to perform a direct addictive-stimulus test.

4. The method according to claim 3, wherein said likelihood information being a set of likelihoods, one for each of a set of stimulus categories, and where each likelihood is associated with a respective threshold, wherein said direct addictive-stimulus test being a test of the stimulus category of which the likelihood exceeds the associated threshold.

5. The method according to claim 4, wherein said requesting said individual to perform a direct addictive-stimulus test comprises a time when said individual is to perform said direct addictive-stimulus test.

6. The method according to claim 5, wherein said time is set within a detection window, from the time of measurement, of said stimulus category.

7. The method according to the claim 1, wherein said comparison comprises a determination of an absolute or relative difference between said measurements of bodily conditions and said individualized baseline measurements and a comparison of said absolute or relative difference with a difference threshold.

8. The method according to claim 1, wherein said eye conditions are selected from:
pupil size;
pupillary light reflex;
behaviour of crossing eyes;
nystagmus;
saccadic eye movements; and
colour of eye whites.

9. The method according to claim 1, wherein said bodily conditions also comprise motion conditions.

10. The method according to claim 9, wherein said motion conditions being selected from:
accelerometer measurements of hand motion; and
stability of camera by analysing collected video filmed by said individual.

11. The method according to claim 1, wherein said bodily conditions are at least two different bodily conditions.

12. The method according to claim 1, wherein said estimating of likelihood information is further based on stored information about historical behaviour of said individual.

13. The method according to claim 1, wherein said at least 5 measurements of eye conditions comprise at least one measurement made in low light ambient conditions of less than 100 lux, and wherein said at least 5 measurements of eye conditions comprise at least one measurement made in bright indoor light ambient conditions of at least 300 lux.

14. The method according to claim 1, wherein said individualization model accounts for acquired skills.

15. The method according to claim 14, wherein said accounting for acquired skills comprises one of:
disregarding the initial attempts that could be of underperforming nature if the skill was not yet acquired; and
relying on a fraction of the strongest results indicating the acquired skill.

16. The method according to claim 15, wherein said acquired skill is the ability to cross eyes.

17. A system for self-administrated surveillance of use of addictive-stimulus for an individual, comprising a central server and a handheld user interaction device, communicationally connected to each other,
said central server being configured for providing, to said user interaction device, a body-measurement schedule for said individual, said body-measurement schedule comprising multiple measurement time-slots;
said central server being configured for, by means of said user interaction device, requesting said individual to perform measurements of bodily conditions within each of said multiple measurement time-slots;
wherein said bodily conditions comprise eye conditions;
said user interaction device having measurement means, comprising a camera, for collecting data analysable for obtaining each of said measurements of bodily conditions, and a timer for determining a respective time when said measurements of bodily conditions were performed;
said user interaction device being configured for transmitting said collected data from said user interaction device to said central server;
said central server being configured for receiving said collected data and storing said collected data in a memory;
said central server being configured for generating individualized baseline data for an individualization model associated with an individual, said individualization model comprising data of eye conditions analysable from camera recordings when being unexposed to any addictive stimulus;
said generation of said individualization model being based on at least 5 measurements of eye conditions when being unexposed to any addictive stimulus;
said central server comprising a processor being configured for estimating likelihood information of that said individual was exposed to an addictive stimulus, based on at least said collected data;
said estimating comprises comparison of said measurements of bodily conditions with individualized baseline data associated with said individual, said individualized base line data comprising data of said bodily conditions when being unexposed to said addictive stimulus;
said central server being configured for initiating an addictive-stimulus use-discouraging action as a response to data of said likelihood information being larger than a predetermined threshold.

18. The method according to claim 3, wherein said requesting said individual to perform a direct addictive-stimulus test comprises a time when said individual is to perform said direct addictive-stimulus test.

19. The method according to claim 2, wherein said comparison comprises a determination of an absolute or relative difference between said measurements of bodily conditions and said individualized baseline measurements and a comparison of said absolute or relative difference with a difference threshold.

20. The method according to claim 3, wherein said comparison comprises a determination of an absolute or relative difference between said measurements of bodily conditions and said individualized baseline measurements and a comparison of said absolute or relative difference with a difference threshold.
